(19) United States
(12) Patent Application Publication
(10) Pub. No.: US 2026/0060624 A1
(43) Pub. Date: Mar. 5, 2026

(54) RADIATION DETECTOR ASSEMBLY AND MEDICAL IMAGING DEVICE

(71) Applicant: GE Precision Healthcare LLC, Waukesha, WI (US)

(72) Inventors: Xinfeng Li, Beijing (CN); Dongmei Wang, Beijing (CN); Yuwei Zhang, Beijing (CN)

(21) Appl. No.: 19/315,133

(22) Filed: Aug. 29, 2025

(30) Foreign Application Priority Data
Aug. 30, 2024 (CN) 202411210451.8

(51) Int. Cl. A61B 6/42; A61B 6/00; A61B 6/03; A61B 6/10

(52) U.S. Cl. CPC A61B 6/4266; A61B 6/03; A61B 6/107; A61B 6/4488

(57) ABSTRACT

Embodiments of the present application provide a radiation detector assembly and a medical imaging device. The radiation detector assembly includes: a main circuit board, including a first region and a second region, wherein a detector component irradiated by rays is mounted in the first region, and a signal processing component that receives a signal generated by the detector component is mounted in the second region; a thermal control assembly, thermally coupled to the first region of the main circuit board; and a support assembly, supporting the main circuit board and thermally coupled to the second region of the main circuit board.

### CROSS-REFERENCE TO RELATED APPLICATIONS

This application claims priority to Chinese Application No. 202411210451.8, filed on Aug. 30, 2024, the disclosure of which is incorporated herein by reference in its entirety.

### TECHNICAL FIELD

Embodiments of the present application relate to the technical field of imaging devices, and in particular to a radiation detector assembly and a medical imaging device.

### BACKGROUND

Imaging devices are used to scan a subject under examination (such as a patient or a workpiece) in a non-invasive or non-destructive manner, to obtain an internal structure image of an anatomical tissue or site of interest of the subject under examination to assist in diagnosis. An imaging device usually includes a circular scanning bore for a subject under scanning examination to move in or out, and includes a detector mounted along the entire circumference or a partial arc of the circular bore. The detector includes a plurality of detector assemblies mounted on a gantry.

For example, a computed tomography (CT) device is generally used as a medical imaging device for scanning a patient to acquire a tomographic medical image of a site of interest of the patient to assist a doctor in diagnosis. The CT device includes a plurality of detector assemblies that receive X-rays emitted from an X-ray tube and passing through the patient, and forms and quantities of the detector assemblies depend on clinical needs and the design of a CT system.

Each detector assembly of the CT device generally includes a pixelated scintillator and a photoelectric conversion device sequentially arranged along a ray transmission direction. The scintillator is configured to receive the X-rays passing through the patient and generate light. The photoelectric conversion device (such as a photodiode) converts the light generated by the scintillator into an electrical signal. Each detector assembly further includes a collimator configured to collimate the X-rays passing through the patient to a specific direction to avoid or reduce interference between pixels of the scintillator. Each detector assembly further includes a signal circuit board configured to process the electrical signal generated by the photoelectric conversion device, and the like.

The image quality of the CT system depends on various factors such as the detector. Usually, the detector has a good effect when performing calibration and imaging at a known temperature, but the assemblies in the detector are relatively sensitive to temperature. To address the temperature sensitivity of the detector, in the prior art, an active heating manner is often used to heat the photoelectric conversion device in the detector to maintain temperature stability. However, during use, an analog-to-digital converter (ADC), an FPGA chip, a power supply chip, and the like on the circuit board generate a large amount of heat, which affects the temperature of the photoelectric conversion device.

It should be noted that the above introduction of the background is only for the convenience of clearly and completely describing the technical solutions of the present application, and for the convenience of understanding for those skilled in the art.

### SUMMARY

In view of at least one of the aforementioned technical problems or other similar problems, embodiments of the present application provide a radiation detector assembly and a medical imaging device, to perform temperature control on a circuit board in the detector assembly in a partitioned or layered manner, thereby implementing thermal control more accurately and improving the temperature stability of a photoelectric conversion device in the detector assembly.

According to one aspect of the embodiments of the present application, provided is a radiation detector assembly. The radiation detector assembly includes a main circuit board, comprising a first region and a second region, wherein a detector component irradiated by rays is mounted in the first region, and a signal processing component that receives a signal generated by the detector component is mounted in the second region, a thermal control assembly, thermally coupled to the first region of the main circuit board, and a support assembly, supporting the main circuit board and thermally coupled to the second region of the main circuit board.

According to another aspect of the embodiments of the present application, further provided is a radiation detector assembly. The radiation detector assembly includes a main circuit board, comprising a first region, wherein a detector component irradiated by rays is mounted in the first region, an auxiliary circuit board, comprising a first signal processing component, wherein the first signal processing component processes a signal received by the main circuit board, and a support assembly, supporting the main circuit board and separately thermally coupled to the main circuit board and the auxiliary circuit board.

According to another aspect of the embodiments of the present application, provided is a medical imaging device. The medical imaging device is provided with the radiation detector assembly according to the aforementioned aspects.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a CT device according to an embodiment of the present application;

FIG. 2 is a schematic diagram of a CT imaging system according to an embodiment of the present application;

FIG. 3 is a schematic diagram of a radiation detector assembly according to an embodiment of the present application;

FIG. 4 is a schematic diagram of a thermal control assembly according to an embodiment of the present application;

FIG. 5 is a schematic diagram of a support assembly according to an embodiment of the present application; and

FIG. 6 is a schematic diagram of a radiation detector assembly according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “based on” should be construed as “based at least in part on . . . ”, unless otherwise specified in the context.

The features described and/or illustrated for one implementation may be used in one or more other implementations in the same or similar way, be combined with features in other implementations, or replace features in other implementations. The terms “include/comprise” when used herein refer to the presence of features, integrated components, steps, or assemblies, but do not preclude the presence or addition of one or more other features, integrated components, steps, or assemblies.

The medical imaging device described in the present application is applicable to various medical imaging modalities. The medical imaging device includes, but is not limited to, a computed tomography (CT) imaging device, or a positron emission tomography (PET)-CT, or any other suitable medical imaging device.

A system obtaining medical imaging data may include the aforementioned medical imaging device, and may include a separate computer device connected to the medical imaging device, and may further include a computer device connected to an Internet cloud, the computer device being connected by means of the Internet to the medical imaging device or a memory for storing medical images. The imaging method may be independently or jointly implemented by the aforementioned medical imaging device, the computer device connected to the medical imaging device, and the computer device connected to the Internet cloud. For example, the system obtaining the medical image data may be a CT imaging system, etc.

As an example, the embodiments of the present application are described below in conjunction with an X-ray computed tomography (CT) imaging device. Those skilled in the art would appreciate that the embodiments of the present application can also be applied to other medical imaging devices.

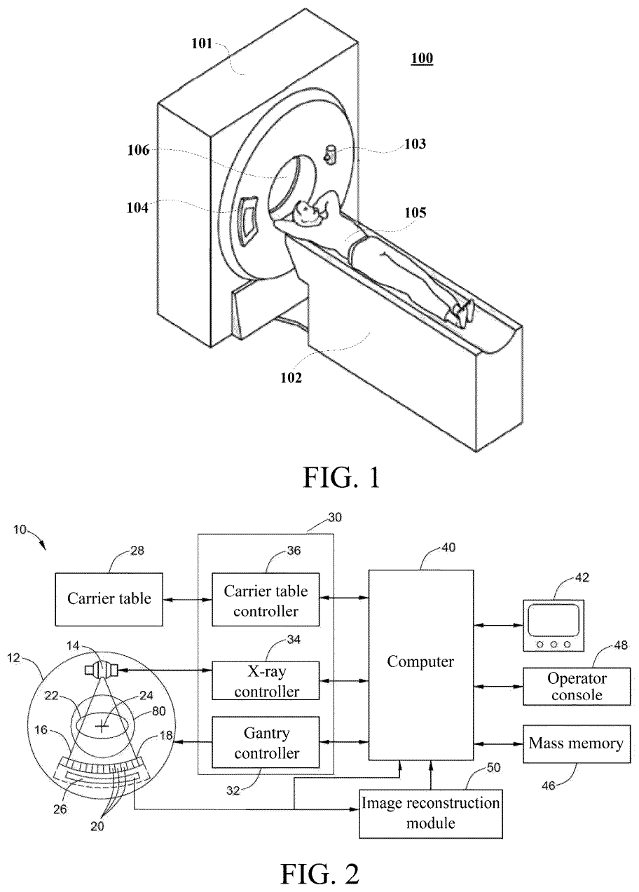

FIG. 1 is a schematic diagram of a CT device according to an embodiment of the present application, and schematically shows a CT device 100. As shown in FIG. 1, the CT device 100 includes a scanning gantry 101 and a patient table 102. The scanning gantry 101 has an X-ray source 103, and the X-ray source 103 projects an X-ray beam toward a detector assembly or collimator 104 on an opposite side of the scanning gantry 101. A subject under examination 105 can lie flat on the patient table 102 and be moved into a scanning gantry opening 106 along with the patient table 102. Medical image data of the subject under examination 105 can be obtained by scanning performed by the X-ray source 103.

FIG. 2 is a schematic diagram of a CT imaging system according to an embodiment of the present application, and schematically shows a block diagram of a CT imaging system 10. As shown in FIG. 2, the system 10 includes a scanning gantry 12. An X-ray source 14 and a detector 18 are arranged opposite to each other on the scanning gantry 12. The detector 18 is composed of a plurality of detector assemblies 20 and a data acquisition system (DAS) 26. The DAS 26 is configured to convert sampled analog data of analog attenuation data received by the plurality of detector assemblies 20 into digital signals for subsequent processing.

In some embodiments, the system 10 is used to acquire, from different angles, projection data of a subject to be examined. Thus, components on the gantry 12 are used to rotate around a rotation center 24 to acquire projection data. During rotation, the X-ray radiation source 14 is used to emit toward the detector 18 X-rays 16 that pass through the subject to be examined. Attenuated X-ray beam data is preprocessed and then used as projection data of a target volume of the subject. An image of the subject to be examined may be reconstructed on the basis of the projection data. The reconstructed image may display internal features of the subject to be examined. These features include, for example, a lesion, the size, the shape, etc., of a body tissue structure. The center of rotation 24 of the gantry also defines the center of a scanning field 80.

In some embodiments, the system 10 includes a control mechanism 30. The control mechanism 30 may include an X-ray controller 34 used to provide power and timing signals to the X-ray radiation source 14. The control mechanism 30 may further include a gantry controller 32 used to control the rotational speed and/or position of the gantry 12 on the basis of imaging requirements. The control mechanism 30 may further include a carrier table controller 36 which is configured to drive a carrier table 28 to move to a suitable position, so as to position the subject to be examined in the gantry 12 to perform a scout scan or an axial scan or a bolt scan or other scan modes in order to acquire the projection data of the target volume of the subject to be examined. Furthermore, the carrier table 28 includes a driving apparatus, and the carrier table controller 36 may control the carrier table 28 by controlling the driving apparatus.

The system 10 further includes an image reconstruction module 50. As described above, the DAS 26 samples and digitizes the projection data acquired by the plurality of detector assemblies 20. Next, the image reconstruction module 50 performs high-speed image reconstruction on the basis of the aforementioned sampled and digitized projection data. In some embodiments, the image reconstruction module 50 stores the reconstructed image in a storage device or a mass memory 46. Or, the image reconstruction module 50 transmits the reconstructed image to a computer 40 to generate information for diagnosing and evaluating patients. For example, a first scout image and a diagnostic section image are generated on the basis of the projection data acquired by the scout scan or the axial scan or the bolt scan or the other scan modes.

Although the image reconstruction module 50 is illustrated as a separate entity in FIG. 2, in some embodiments, the image reconstruction module 50 may form a part of the computer 40. Or, the image reconstruction module 50 may not exist in the system 10, or the computer 40 may perform one or more functions of the image reconstruction module 50. Furthermore, the image reconstruction module 50 may be located at a local or remote location, and may be connected to the system 10 using a wired or wireless network. In some embodiments, computing resources having a centralized cloud network may be used for the image reconstruction module 50.

In some embodiments, the system 10 further includes the computer 40, wherein data sampled and digitized by the DAS 26 and/or an image reconstructed by the image reconstruction module 50 is transmitted to a computer or the computer 40 for processing. In some embodiments, the computer 40 stores the data and/or image in a storage device such as a mass memory 46. The mass memory 46 may include a hard disk drive, a floppy disk drive, a CD-read/write (CD-R/W) drive, a digital versatile disc (DVD) drive, a flash drive, and/or a solid-state storage apparatus. A processor in the computer 40 determines the predicted section image according to the first scout image.

In some embodiments, the computer 40 transmits the reconstructed image and/or other information to a display 42, the display 42 being communicatively connected to the computer 40 and/or the image reconstruction module 50. The computer 40 may be connected to a local or remote display, printer, workstation and/or similar device, for example, connected to such devices of medical institutions or hospitals, or connected to a remote device by means of one or a plurality of configured wires or a wireless network such as the Internet and/or a virtual private network. For example, the display displays the predicted section image and the corresponding scanning parameters.

Furthermore, the computer 40 may provide commands and parameters to the DAS 26 and the control mechanism 30 (including the gantry controller 32, the X-ray controller 34, and the carrier table controller 36) on the basis of user provision and/or system definition, so as to control a system operation, such as data acquisition and/or processing. In some embodiments, the computer 40 controls the system operation on the basis of user input. For example, the computer 40 may receive user input such as commands, scanning protocols and/or scanning parameters, by means of an operator console 48 connected thereto. The operator console 48 may include a keyboard (not shown) and/or touch screen to allow a user to input/select commands, scanning protocols and/or scanning parameters.

In some embodiments, the system 10 may include or be connected to a picture archiving and communication system (PACS) (not shown in the figure). In some embodiments, the PACS is further connected to a remote system such as a radiology information system, a hospital information system, and/or an internal or external network (not shown) to allow operators at different locations to provide commands and parameters and/or access image data.

The computer 40 may be configured and/or arranged for use in different manners. For example, in some implementations, a single computer 40 may be used; and in other implementations, a plurality of computers 40 are configured to work together (for example, on the basis of a distributed processing configuration) or separately, and each computer 40 is configured to process specific aspects and/or functions, and/or process data for generating models used only for a specific system 10. In some implementations, the computer 40 may be local (for example, in the same place as one or a plurality of systems 10, for example, in the same facility and/or the same local network); and in other implementations, the computer 40 may be remote and thus only accessible by means of a remote connection (for example, by means of the Internet or other available remote access technologies). In a specific implementation, the computer 40 may be configured in a manner similar to that of cloud technology, and may be accessed and/or used in a manner substantially similar to that of accessing and using other cloud-based systems.

The device and system for acquiring medical image data (which may also be referred to as medical images or medical image data) according to the embodiments of the present application are schematically described above, but the present application is not limited thereto. The medical imaging device may be a CT device, a PET-CT, or any other suitable imaging device. The storage device may be located in the medical imaging device, in a server outside the medical imaging device, in an independent medical imaging storage system (such as a picture archiving and communication system (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SaaS)”. SaaS can exist between hospitals, between a hospital and an imaging center, or between a hospital and a third-party online diagnosis and treatment service provider.

Medical image scanning is schematically illustrated above, and the embodiments of the present application are described in detail below in connection with the drawings. In the embodiments described below, the medical imaging device being a CT device is used as an example for description, and the content of the description is also applicable to other medical imaging devices. The detector in the CT device is composed of a plurality of radiation detector assemblies. For example, the plurality of radiation detector assemblies may have a flat panel form factor. The plurality of radiation detector assemblies are mounted on a guide rail. The guide rail is integrally formed and has a plate-like structure with a certain curvature, and may be mounted on a gantry (which is also referred to as a rotating gantry). The flat panel form factor of each radiation detector assembly means that the size of a radiation receiving plane of the radiation detector assembly that receives rays or faces a radiation source is much larger than or several times the size of the radiation detector assembly parallel to a ray propagation path. For example, the length or width of the radiation detector assembly with a rectangular radiation receiving plane is much greater than the thickness thereof. The plurality of radiation detector assemblies are sequentially arranged on the guide rail along the extension direction of the guide rail, and are arranged on the guide rail in a certain mounting manner, for example, in a mechanical mounting manner by means of fixing holes provided in the guide rail. As described above, the plurality of radiation detector assemblies are arranged on the gantry by being mounted on the guide rail, and rotate with movement of the gantry. In the following embodiments, one of the radiation detector assemblies is used as an example for description, but the embodiments of the present application are not limited thereto.

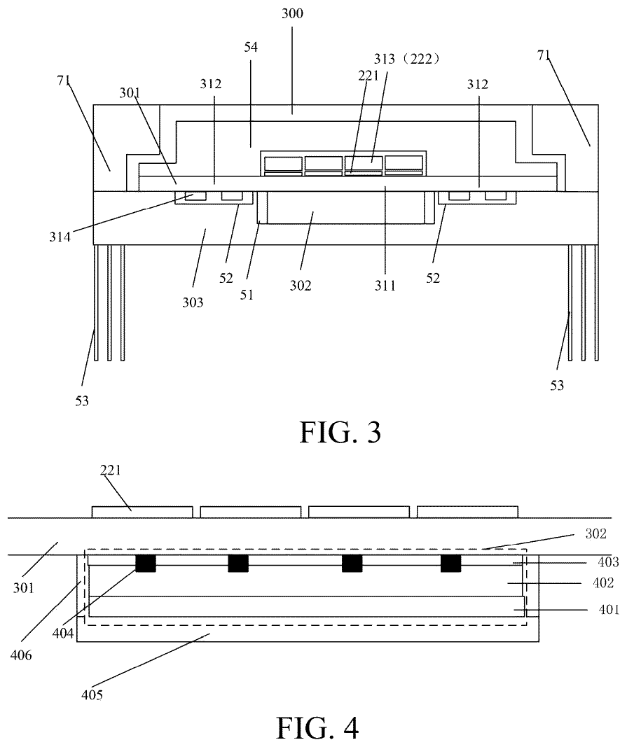

An embodiment of the present application provides a radiation detector assembly. FIG. 3 is a schematic diagram of a radiation detector assembly according to an embodiment of the present application. Other radiation detector assemblies in the CT device are similar thereto in structure. As shown in FIG. 3, the radiation detector assembly 300 includes a main circuit board 301, including a first region 311 and a second region 312, wherein a detector component 313 irradiated by rays is mounted in the first region 311, and a signal processing component 314 that receives a signal generated by the detector component 313 is mounted in the second region 312. FIG. 3 also includes a thermal control assembly 302, thermally coupled to the first region 311 of the main circuit board 301 and a support assembly 303, supporting the main circuit board 301 and thermally coupled to the second region 312 of the main circuit board 301.

In some embodiments, the main circuit board 301 may be, for example, a printed circuit board. The detector component 313 irradiated by rays may include a photoelectric conversion device 221 (for example, a photodiode) and a scintillator 222. The scintillator has a plurality of scintillator pixels configured to receive attenuated rays for converting the rays into visible light. The photoelectric conversion device 221 is optically coupled to the scintillator and configured to detect light outputted from the corresponding scintillator pixels and convert the light into an electrical signal. The detector component 313 may be located on one side (an upper side in FIG. 3) of the main circuit board 301, that is, a side of the main circuit board that is directly irradiated by rays. The signal processing component 314 that receives the signal generated by the detector component 313 includes an application-specific integrated chip (ASIC), a field programmable gate array (FPGA), a power supply chip, an analog-to-digital converter (ADC), and the like, and may be located on the other side (a lower side in FIG. 3) of the main circuit board 301, that is, the side of the main circuit board 301 that is not directly irradiated by rays. The thermal control assembly 302, the support assembly 303, and the signal processing component 314 are located on the same side of the main circuit board 301 that is not directly irradiated by rays. As described above, the radiation detector assembly 300 may have a flat panel form factor, and correspondingly, the main circuit board 301 also has a flat panel appearance (not shown in a top view), especially a flat panel appearance with a large area, so that a large number of detector components 313 may be mounted on a circuit board of the main circuit board 301 or the detector components 313 may be mounted at a high density on the circuit board of the main circuit board 301, thereby reducing costs of the radiation detector assembly 300. In addition, the main circuit board 301 is parallel to the support assembly 303, so that the overall height of the radiation detector assembly 300 can be reduced, and the flat panel form factor of the radiation detector assembly 300 can be implemented.

In some embodiments, the first region 311 and the second region 312 are non-overlapping regions. The first region 311 may be one or a plurality of regions that are communicated or non-communicated. The second region 312 may be one or a plurality of regions that are communicated or non-communicated. As shown in FIG. 3, the first region 311 is a region located in the middle of the main circuit board 301, and the second region 312 is two non-communicated regions located on two sides of the first region 311. Here is only an example, and the embodiments of the present application are not limited thereto. For example, the first region may be a plurality of non-communicated regions, which will not be illustrated one by one here.

In some embodiments, the detector component 313 is mounted in the first region 311, and the thermal control assembly 302 is thermally coupled to the first region 311, that is, the thermal control assembly 302 performs thermal control on the detector component 313, for example, heats the detector component 313. The signal processing component 314 is mounted in the second region 312. The support assembly 303 is thermally coupled to the second region 312. Since the signal processing component 314 generates a large amount of heat, the support assembly 303 may dissipate heat from the signal processing component 314. That is, the thermal control assembly 302 and the support assembly 303 perform temperature control in a partitioned manner, thereby implementing thermal control more accurately and improving the temperature stability of the photoelectric conversion device in the detector assembly. The thermal control assembly 302 and the support assembly 303 are described in detail below.

FIG. 4 is a schematic diagram of the thermal control assembly 302 according to an embodiment of the present application. As shown in FIG. 4, the thermal control assembly 302 includes an electric heater 401 and a thermally conductive component 402 thermally coupled to the electric heater 401 and the first region 311 of the main circuit board 301. The electric heater 401 may be a thin-film heating resistor, which is connected to an external power source through a power line and generates heat by converting electrical energy into thermal energy. The thermally conductive component 402 is separately thermally coupled to the electric heater 401 and the first region 311. The detector component 313 and the thermally conductive component 402 are both located in the first region, but are respectively located on two sides of the main circuit board 301. The thermally conductive component 402 may transfer heat generated by the electric heater 401 to the detector component 313 mounted in the first region 311. The thermally conductive component 402 may be made of an aluminum or copper material, and the embodiments of the present application are not limited thereto. The thermally conductive component 402 may further prevent electromagnetic interference.

In some embodiments, optionally, the thermally conductive component 402 may further include a plate-like structure made of a thermally conductive metallic material (for example, aluminum or copper). Due to the plate-like structure, the heat generated by the electric heater 401 may be uniformly transferred to the detector component 313 corresponding to the first region.

In some embodiments, optionally, a radiation shielding component 403 is further arranged between the thermally conductive component 402 and the first region of the main circuit board 301. The radiation shielding component 403 may be of a plate-like structure made of tungsten or lead or a lead alloy or a tungsten alloy, and is configured to absorb rays penetrating the main circuit board 301, to improve the imaging quality of an image and avoid the generation of artifacts in the image caused by the circuit board or the like.

In some embodiments, when the detector component needs to be heated, the electric heater 401 may be controlled by a controller, and the controller may adjust a heating temperature of the electric heater 401. Optionally, the thermal control assembly 302 further includes a thermal sensor 404 mounted to the thermally conductive component 402. That is, the thermal sensor 404 may be located in the first region. Specifically, the thermal sensor may be located on the front side or back side of the detector component on the main circuit board 301, and is thermally coupled to the detector component by means of the main circuit board 301, to accurately detect the temperature of the detector component. There may be one or a plurality of thermal sensors 404, configured to detect the actual temperature of the detector component. The controller may further control the heating temperature of the electric heater of the thermal control assembly 302 according to the temperature detected by the thermal sensor 404, so that the temperature of the detector component is maintained at a preset operating temperature.

In some embodiments, the thermal control assembly 302 may, by means of a thermally conductive part (such as a thermally conductive adhesive), be fixed on the side of the first region 311 of the main circuit board 301 that is not directly irradiated by rays.

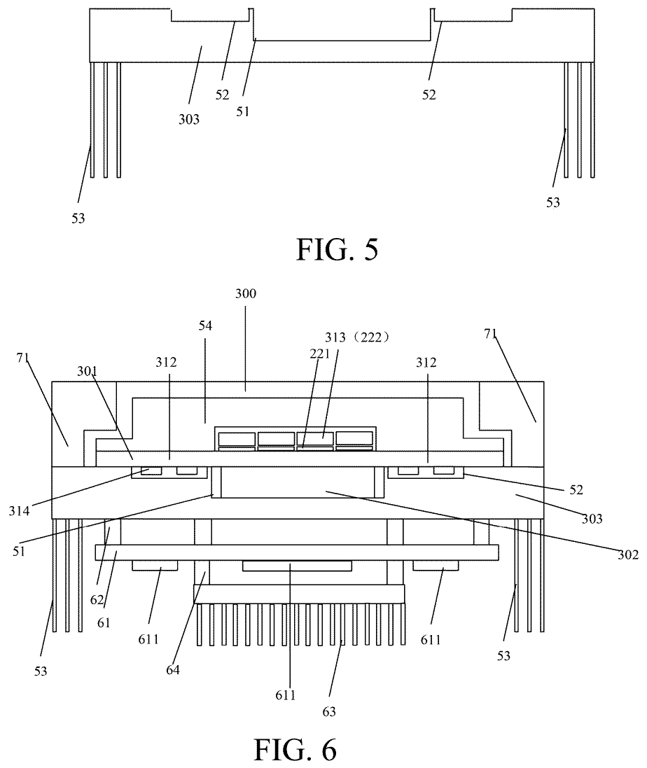

FIG. 5 is a schematic diagram of the support assembly 303 according to an embodiment of the present application. FIG. 5 shows an end view or a sectional view of the support assembly 303, wherein the support assembly 303 is approximately strip-shaped, and is located on the side of the main circuit board 301 that is not directly irradiated by rays. The support assembly 303 is slightly longer than the main circuit board 301, and may be connected to the main circuit board 301 and the signal processing component 314 by means of a thermally conductive part (such as a thermally conductive adhesive). The support assembly 303 may be made of a thermally conductive metallic material, such as an aluminum or aluminum alloy material, and the support assembly 303 can release heat generated by the signal processing component 314 into the surrounding air. As described above, the radiation detector assembly 300 may have a flat panel form factor, and correspondingly, the support assembly 303 also has a substantially flat panel form factor (not shown in a top view). A main body part (for example, a first accommodating space 51 described below) of the support assembly 303 is parallel to the main circuit board 301, so that the overall height of the radiation detector assembly 300 can be reduced, and the flat panel form factor of the radiation detector assembly 300 can be implemented.

In some embodiments, as shown in FIG. 3, the radiation detector assembly 300 may be mounted on a guide rail 71 using the support assembly 303, and two ends of the support assembly 303 are fixedly connected to the guide rail 71. The support assembly 303 supports the main circuit board 301 or the radiation detector assembly 300.

In some embodiments, as shown in FIG. 5, the support assembly 303 is provided with the first accommodating space 51, and the thermal control assembly 302 is located in the first accommodating space 51. That is, the support assembly 303 is provided with the first accommodating space 51 at a position close to the first region of the main circuit board 301. The first accommodating space 51 may include one or more accommodating spaces that are communicated or non-communicated, the specific position of which may correspond to the first region. For example, when the first region includes a plurality of non-communicated regions, the first accommodating space 51 also includes a plurality of non-communicated accommodating spaces that are in a one-to-one correspondence with the plurality of regions of the first region. Each accommodating space accommodates the aforementioned thermal control assembly 302.

In some embodiments, as shown in FIG. 5, the support assembly 303 is provided with a second accommodating space 52 at a connection point between the support assembly 303 and the main circuit board 301, and the signal processing component 314 is located in the second accommodating space 52. The support assembly 303 is connected to the signal processing component 314 using a thermally conductive part (such as a thermally conductive adhesive). The second accommodating space 52 may include one or a plurality of accommodating spaces that are communicated or non-communicated, the specific position of which may correspond to the second region. For example, when the second region includes a plurality of non-communicated regions, the second accommodating space 52 also includes a plurality of non-communicated accommodating spaces that are in a one-to-one correspondence with the plurality of regions of the second region. Each accommodating space accommodates one or a plurality of signal processing components 314. In addition, the first accommodating space 51 and the second accommodating space 52 are not communicated with each other.

The inventors have also found that in conventional CT detectors, a large amount of heat generated by a heater is released to the outside of a detector through a cooling surface and carried away by the surrounding air because both heat dissipation (cooling) and heating exist simultaneously. Consequently, the detector has to increase the power of the heater to compensate for heat dissipation losses. In the present embodiment of the present application, a thermal insulation component 405 is arranged between the thermal control assembly 302 and the support assembly 303. The thermal insulation component 405 is arranged to thermally isolate the thermal control assembly 302 from the support assembly 303 that has a thermal dissipation function. Therefore, the waste of electric energy caused by simultaneous heating and cooling of the detector during a temperature control process is reduced.

In some embodiments, the thermal control assembly 302 is contained in the first accommodating space 51, but the thermal control assembly 302 is not thermally coupled to an inner wall of the first accommodating space 51. For example, as shown in FIG. 4, the bottom of the thermal control assembly 302 is fixedly connected to a bottom surface of the inner wall of the first accommodating space 51 of the support assembly 303 by means of the thermal insulation component 405 (for example, a thermal insulation adhesive). The signal processing component 314 is contained in the second accommodating space 52, but the signal processing component 314 does not need to be thermally insulated from the second accommodating space 52. Therefore, heat generated during operation of the signal processing component 314 may be released to the surrounding air through the support assembly 303. However, since the thermal control assembly 302 is thermally isolated from the support assembly 303, heat generated by heating of the thermal control assembly 302 is not released to the outside of the detector assembly 300 through the support assembly in a large amount and carried away by the surrounding air. Therefore, the efficiency of the electric heater 401 is improved, and energy consumption is reduced. In addition, the bottom of the thermal control assembly 302 is fixedly connected to the bottom surface of the inner wall of the first accommodating space 51 of the support assembly 303 by means of the thermal insulation adhesive, which can further support the thermal control assembly 302 and prevent the detector component (for example, the photoelectric conversion device) from being deformed when the gantry rotates.

In some embodiments, the thermal control assembly 302 in the first accommodating space 51 of the support assembly is at least partially thermally isolated from the signal processing component 314 in the second accommodating space 52. For example, gaps are reserved between side surfaces of the inner wall of the first accommodating space 51 and two sides of the thermal control assembly 302. The gaps may be filled with a medium such as air having a low thermal conductivity, for example, a thermal insulation material. Therefore, heat generated by heating of the thermal control assembly 302 is not released to the outside of the detector through the support assembly in a large amount and carried away by the surrounding air. Therefore, the efficiency of the electric heater 401 is improved, and energy consumption is reduced. In some embodiments, the thermal control assembly 302 is accommodated in the first accommodating space 51 and the signal processing component 314 is accommodated in the second accommodating space 52, which can further reduce the overall height of the radiation detector assembly 300 and implement the flat panel form factor of the radiation detector assembly 300.

In some embodiments, as shown in FIG. 5, the support assembly 303 is further provided with a first heat sink 53. The first heat sink 53 may be arranged on the side of the support assembly opposite to the side of the support assembly that is connected to the main circuit board 301. For example, the first heat sink is arranged at two ends of the support assembly, which is only an example here. For example, the first heat sink 53 may alternatively be arranged in the middle of the support assembly 303, or arranged on both side surfaces of the support assembly 303 in the width direction. The embodiments of the present application are not limited thereto. Due to the provision of the first heat sink 53, a heat dissipation effect of the support assembly 303 may be further improved.

In some embodiments, the radiation detector assembly 300 may further include a collimator 54. The collimator 54 is arranged on a surface of the detector component 313. The collimator 54 collimates rays emitted by a radiation source to the detector component 313. There is no limitation on implementations of the collimator 54.

FIG. 6 is a schematic diagram of a radiation detector assembly according to an embodiment of the present disclosure, which differs from FIG. 3 in that the radiation detector assembly 300 further includes an auxiliary circuit board 61, the auxiliary circuit board 61 is in communication with the signal processing component 314 of the main circuit board 301, and one side of the auxiliary circuit board 61 is thermally coupled to the support assembly 303. That is, the support assembly 303 is separately thermally coupled to the main circuit board 301 and the auxiliary circuit board 61, so that temperature control is performed in a layered manner, thereby implementing thermal control more accurately.

In some embodiments, a signal processing component 611 (hereinafter, the signal processing component 611 is referred to as a first signal processing component, and the signal processing component 314 is referred to as a second signal processing component) is mounted on the auxiliary circuit board 61. The first signal processing component may also include an application-specific integrated chip (ASIC), a field programmable gate array (FPGA), a power supply chip, an analog-to-digital converter (ADC), and the like. The first signal processing component is in communication with the second signal processing component. The first signal processing component may receive, via the second signal processing component, a signal generated by the detector component, or receive a signal processed by the second signal processing component. The first signal processing component is mounted on the other side of the auxiliary circuit board 61. As mentioned above, the radiation detector assembly 300 may have a flat panel form factor, and correspondingly, the auxiliary circuit board 61 also has a substantially flat panel form factor (not shown in a top view), on which signal processing components 611 may be mounted at a high density. As shown in FIG. 6, the auxiliary circuit board 61 is parallel to the support assembly 303, so that the overall height of the radiation detector assembly 300 can be reduced.

In some embodiments, the auxiliary circuit board 61 is thermally coupled to the support assembly 303 by means of a first connecting part 62 that is thermally conductive.

In some embodiments, to further improve a heat dissipation capability, the radiation detector assembly further includes a second heat sink 63. The other side of the auxiliary circuit board 61 is thermally coupled to the second heat sink 63, for example, thermally coupled to the second heat sink 63 by means of a second connecting part 64 that is thermally conductive. As shown in FIG. 6, the second heat sink 63 slightly extends beyond heat sink fins of the first heat sink 53 in the extension direction of heat sink fins thereof. In other implementations, the second heat sink 63 does not extend beyond the first heat sink 53 in the extension direction of the heat sink fins thereof, so that the overall height of the radiation detector assembly 300 can be reduced.

It can be learned from the aforementioned embodiments that the thermal control assembly 302 and the support assembly 303 perform temperature control in a partitioned manner, thereby implementing thermal control more accurately and improving the temperature stability of the photoelectric conversion device in the detector assembly 300.

In addition, the support assembly 303 is separately thermally coupled to the main circuit board 301 and the auxiliary circuit board 61, so that temperature control is performed in a layered manner, thereby implementing thermal control more accurately.

In addition, the thermal insulation component 405 is arranged to thermally isolate the thermal control assembly 302 from the support assembly 303 that has a thermal dissipation function. Therefore, the waste of electric energy caused by simultaneous heating and cooling of the detector assembly 300 during a temperature control process is reduced. In addition, the thermal insulation component 405 may further support the thermal control assembly 302, and prevent the detector component (for example, the photoelectric conversion device) from being deformed when the gantry rotates.

In addition, the thermal control assembly 302 in the first accommodating space 51 of the support assembly 303 is at least partially thermally isolated from the signal processing component 314 in the second accommodating space 52, thereby reducing the waste of electric energy caused by simultaneous heating and cooling of the detector assembly 300 during a temperature control process.

An embodiment of the present application further provides a radiation detector assembly (not shown in the figures). The radiation detector assembly includes: a main circuit board, including a first region, wherein a detector component irradiated by rays is mounted in the first region; an auxiliary circuit board, including a first signal processing component, wherein the first signal processing component processes a signal received by the main circuit board; and a support assembly, supporting the main circuit board and separately thermally coupled to the main circuit board and the auxiliary circuit board. For implementations of the main circuit board and the auxiliary circuit board, reference may be made to the aforementioned embodiments, which will not be repeated here. The support assembly is separately thermally coupled to the main circuit board and the auxiliary circuit board, so that temperature control is performed in a layered manner, thereby implementing thermal control more accurately. For example, the support assembly may be thermally coupled to the main circuit board by means of a thermally conductive part (for example, fixedly connected to the main circuit board by means of a thermally conductive adhesive), and thermally coupled to the auxiliary circuit board by means of a first connecting part that is thermally conductive. The support assembly may be of a strip-shaped structure, wherein for the support assembly, reference may be made to the support assembly in the aforementioned embodiment; or may be of another structure that can be separately thermally coupled to the main circuit board and the auxiliary circuit board, and the embodiments of the present application are not limited thereto.

In some embodiments, optionally, the radiation detector assembly further includes: a thermal control assembly, thermally coupled to the first region of the main circuit board.

In some embodiments, optionally, a thermal insulation component is arranged between the thermal control assembly and the support assembly.

In some embodiments, the support assembly is provided with a first accommodating space 51, and the thermal control assembly is located in the first accommodating space. For an implementation of the thermal control assembly, reference may be made to the aforementioned embodiments, which will not be repeated here.

In some embodiments, optionally, the main circuit board further includes a second region in which a second signal processing component is mounted. The support assembly is provided with a second accommodating space at a connection point between the support assembly and the main circuit board, and the second signal processing component is located in the second accommodating space. The thermal control assembly in the first accommodating space of the support assembly is at least partially thermally isolated from the second signal processing component in the second accommodating space. For a specific implementation, reference may be made to the aforementioned embodiments, which will not be repeated here.

An embodiment of the present application further provides a medical imaging device. The medical imaging device of the present application is, for example, a computed tomography (CT) imaging device, a PET-CT, or any other suitable medical imaging device. The medical imaging device includes a radiation detector assembly. For an implementation of the radiation detector assembly, reference may be made to the radiation detector assembly in the aforementioned embodiments, which will not be repeated here. The medical imaging device projects rays emitted by a ray source onto a subject under examination. The radiation detector assembly receives rays attenuated by the subject under examination, converts the rays into an electrical signal, and performs tomographic imaging of the subject under examination according to the electrical signal generated by the radiation detector assembly. The medical imaging device may further include other assemblies. For details, reference may be made to FIG. 2, which will not be repeated here.

The present application is described above with reference to specific implementations. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A radiation detector assembly, comprising:
a main circuit board, comprising a first region and a second region, wherein a detector component irradiated by rays is mounted in the first region, and a signal processing component that receives a signal generated by the detector component is mounted in the second region;
a thermal control assembly, thermally coupled to the first region of the main circuit board; and
a support assembly, supporting the main circuit board and thermally coupled to the second region of the main circuit board.

2. The radiation detector assembly according to claim 1, wherein a thermal insulation component is arranged between the thermal control assembly and the support assembly.

3. The radiation detector assembly according to claim 2, wherein the support assembly is provided with a first accommodating space, and the thermal control assembly is located in the first accommodating space.

4. The radiation detector assembly according to claim 3, wherein the support assembly is provided with a second accommodating space at a connection point between the support assembly and the main circuit board, and the signal processing component is located in the second accommodating space.

5. The radiation detector assembly according to claim 4, wherein the thermal control assembly in the first accommodating space of the support assembly is at least partially thermally isolated from the signal processing component in the second accommodating space.

6. The radiation detector assembly according to claim 5, wherein the thermal control assembly comprises an electric heater and a thermally conductive component thermally coupled to the electric heater and the first region of the main circuit board.

7. The radiation detector assembly according to claim 6, wherein a radiation shielding component is arranged between the thermally conductive component and the first region of the main circuit board.

8. The radiation detector assembly according to claim 6, wherein the thermal control assembly comprises a thermal sensor mounted to the thermally conductive component.

9. The radiation detector assembly according to claim 8, wherein the thermal control assembly, the signal processing component, and the support assembly are located on a same side of the main circuit board not directly irradiated by rays.

10. The radiation detector assembly according to claim 9, wherein the support assembly is provided with a first heat sink.

11. The radiation detector assembly according to claim 1, further comprising an auxiliary circuit board, wherein the auxiliary circuit board is in communication with the signal processing component of the main circuit board, and one side of the auxiliary circuit board is thermally coupled to the support assembly.

12. The radiation detector assembly according to claim 11, further comprising a second heat sink, wherein the other side of the auxiliary circuit board is thermally coupled to the second heat sink.

13. A radiation detector assembly, comprising:
a main circuit board, comprising a first region, wherein a detector component irradiated by rays is mounted in the first region;
an auxiliary circuit board, comprising a first signal processing component, wherein the first signal processing component processes a signal received by the main circuit board; and
a support assembly, supporting the main circuit board and separately thermally coupled to the main circuit board and the auxiliary circuit board.

14. The radiation detector assembly according to claim 13, further comprising a thermal control assembly, thermally coupled to the first region of the main circuit board.

15. The radiation detector assembly according to claim 14, wherein a thermal insulation component is arranged between the thermal control assembly and the support assembly.

16. The radiation detector assembly according to claim 14, wherein the support assembly is provided with a first accommodating space, and the thermal control assembly is located in the first accommodating space.

17. The radiation detector assembly according to claim 16, wherein the thermal control assembly comprises an electric heater and a thermally conductive component thermally coupled to the electric heater and the first region of the main circuit board.

18. The radiation detector assembly according to claim 17, wherein a radiation shielding component is arranged between the thermally conductive component and the first region of the main circuit board.

19. The radiation detector assembly according to claim 18, wherein the thermal control assembly comprises a thermal sensor mounted to the thermally conductive component.

20. The radiation detector assembly according to claim 19, wherein the thermal control assembly, the first signal processing component, and the support assembly are located on a same side of the main circuit board not directly irradiated by rays.
